(19) United States
(12) Patent Application Publication
(10) Pub. No.: US 2014/0323818 A1
(43) Pub. Date: Oct. 30, 2014

(54) Wireless Physiological Monitoring Device

(71) Applicants: Melanie Axelgaard, Fallbrook, CA (US); Inge Axelgaard, Fallbrook, CA (US)

(72) Inventors: Melanie Axelgaard, Fallbrook, CA (US); Inge Axelgaard, Fallbrook, CA (US)

(21) Appl. No.: 13/870,701

(22) Filed: Apr. 25, 2013

(51) Int. Cl. A61B 5/00

(52) U.S. Cl. CPC A61B 5/0022; A61B 5/742; A61B 5/6824
USPC 600/301

(57) ABSTRACT

A wireless physiological signal monitoring device for measuring physiological signals such as body temperature, blood pressure, ECG, heart rate, sleeping patterns and the like. The data that is collected by the device is accessible both on the visual interface of the device as well as on connected devices. The data that is collected by the device sensors is automatically uploaded to a secure cloud server, or alternatively is uploaded to specified external devices. The monitoring device of the invention uses wireless signals to upload the data to the secure cloud server thereby making the real-time data readily accessible regardless of the physical location of the device.

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The present invention relates generally to patient monitoring devices. More particularly the invention concerns a wireless physiological signal monitoring device having an identification system that is both digital and physical.

2. Description of Related Art Including Information Disclosed Under 37 CRF 1.97 and 1.98

Numerous types of patient monitoring devices have been suggested in the past. Typically, these devices can be worn by the patient and in some cases include a single or bi-directional wireless communication link for transmitting data from the appliances to a local hub, receiving station or base station server by way of a wireless radio frequency link.

Exemplary of such devices is the device described in U.S. Pat. No. 8,328,718 issued to Tran, which discloses a health monitoring appliance that can operate in a home, a nursing home, or a hospital. In the Tran system, one or more mesh network appliances are provided to enable wireless communication in the home monitoring system. Appliances in the mesh network can include home security monitoring devices, door alarm, window alarm, home temperature control devices, and fire alarm devices, among others. Appliances in the mesh network can be one of multiple portable physiological transducer, such as a blood pressure monitor, heart rate monitor, weight scale, thermometer, spirometer, single or multiple lead electrocardiograph (ECG), a pulse oxymeter, a body fat monitor and a cholesterol monitor. One Tran appliance comprises a patient monitoring device that can be worn by the patient and includes a single or bi-directional wireless communication link for transmitting data from the appliances to the local hub or receiving station or base station server by way of a wireless radio frequency (RF) link using a proprietary or non-proprietary protocol.

Another such prior art device is disclosed in U.S. Publication No. 2007/0073132 issued to Vosch. The Vosch device concerns an apparatus and method for wireless monitoring of a patient, includes a disposable patch having a relatively thin and flexible sealed housing with a surface capable of being releasably attached to the patient's chest; two electrodes positioned spaced apart in the patch, the two electrodes functioning as an EKG lead by detecting electrical impulses from the patient's heart; a temperature sensor positioned in the patch; and a respiration sensor positioned in the patch.

In current clinical settings, hospitals often place multiple plastic bands on a pediatric patient's wrist or ankle for identification purposes such as the patient's name and potential allergies. At the same time, these patients often undergo tests such as ECG monitoring regularly. As will become apparent from the discussion which follows, the device of the present invention will substantially streamline pediatric care by coupling monitoring with identification. The novel device of the invention will create an easier, economical solution that is also more efficient and sanitary.

### BRIEF SUMMARY OF THE INVENTION

It is an object of the present invention to provide a wireless physiological signal monitoring device for measuring physiological signals such as body temperature, blood pressure, ECG, heart rate, sleeping patterns and the like.

Another object of the invention is to provide a monitoring device of the aforementioned character in which the data that is collected by the device is accessible both on the visual interface of the device as well as on connected devices.

Another object of the invention is to provide a monitoring device as described in the preceding paragraphs in which the data that is collected by the device sensors is automatically uploaded to a secure cloud server, or alternatively is uploaded to specified external devices. The monitoring device of the invention uses wireless signals to upload the data to the secure cloud server thereby making the real-time data readily accessible regardless of the physical location of the wristband.

Another object of the invention is to provide a wireless physiological signal monitoring device of the character described that is usable both in health care and in-home settings.

Another object of the invention is to provide a wireless physiological signal monitoring device of the aforementioned character that functions to increase efficiency and quality of care for pediatric patients both in health care and in-home settings.

Another object of the invention is to provide a wireless physiological signal monitoring device of the aforementioned character that is entirely waterproof thereby ensuring that signal reads can continue during water activities, such as bathing, and also allows the device to be easily sanitized through avenues such as soaking in a solution without adversely affecting any component of the device.

Another object of the invention is to provide a wireless physiological signal monitoring device of the aforementioned character in which the connector band is provided within a slot where physical identification data such as warnings for patient allergies and barcode data can be placed.

Another object of the invention is to provide a monitoring device of the character described that includes a digital touchscreen interface.

Another object of the invention is to provide a monitoring device of the character described that is specially configured for use with infants, preemies and small children.

Another object of the invention is to provide a monitoring device of the character described in the preceding paragraph that is of a layered, pliable design which conforms to the contours of the patient and one that can be moved without interfering with signal readouts or legibility of the visual interface.

Another object of the invention is to provide a monitoring device of the character described in the preceding paragraphs that includes a microphone that is monitored by the system and allows the user to continuously listen to determine if the patient is crying or if there are unusual extraneous noises.

Another object of the invention is to provide a monitoring device of the character described that includes a speaker adapted to play selected sounds, such as lullabies that are uploaded through the cloud system.

Another object of the invention is to provide a monitoring device of the character described that measures physiological signals as well as external factors such as room temperature.

Another object of the invention is to provide a monitoring device of the character described that can be plugged into mobile devices and traditional ECG monitors.

Another object of the invention is to provide a monitoring device of the character described that can be worn either on the patient's arm or leg and one that can be readily adjusted to accommodate patients of different ages, sizes and physical condition.

Another object of the invention is to provide a monitoring device of the character described that includes strategically located continuous light sources that can be programmed to display colors, including pink or blue to represent the patient's gender, green or red to indicate whether the patient's vitals are normal or cause for concern as well as other selected colors to indicate a patient affliction such as infant jaundice, allergies and the like.

Another object of the invention is to provide a monitoring device of the aforementioned character that includes a combination ultraviolet (UV), infrared (IV) protective film that is superimposed over the sensors that monitor the patient's vital signs. This protective film functions to protect the sensors of the device from harmful radiation so that they will effectively operate in virtually any operational environment and can at all times accurately determine the patient's temperature even in an incubator.

Another object of the invention is to provide a monitoring device of the aforementioned character that enables real time data to be continuously reviewed and one that includes thresholds that are preprogrammed into the system, or custom thresholds that can be added by the user. The monitoring device also enables the continuous review of historical data and provides alerts if problematic or unusual trends are observed.

These and other objects of the invention will be realized by the device construction illustrated in the drawings and described in the paragraphs that follow.

### BRIEF DESCRIPTION OF SEVERAL VIEWS OF THE DRAWINGS

FIG. 1 is a generally perspective view of one form of the wireless physiological signal monitoring device of the invention.

FIG. 2 is a cross-sectional view taken along lines 2-2 of FIG. 1.

FIG. 3 is a top plan view of the monitoring device shown in FIG. 1.

FIG. 4 is a bottom plan view of the monitoring device shown in FIG. 1.

FIG. 5A is a generally perspective, exploded view of the monitoring device illustrated in FIG. 1.

FIG. 5B is a generally perspective, exploded view of the vital sign monitor assembly of the monitoring device illustrated in FIG. 1.

FIG. 6 is a generally diagrammatic view, illustrating one form of the method of operation of the monitoring device of the invention.

FIGS. 7A through 7D diagrammatically illustrate several alternate constructions of the monitoring device of the invention.

FIG. 8 is a generally diagrammatic view, further illustrating the method of operation of the monitoring device of the invention.

### DETAILED DESCRIPTION OF THE INVENTION

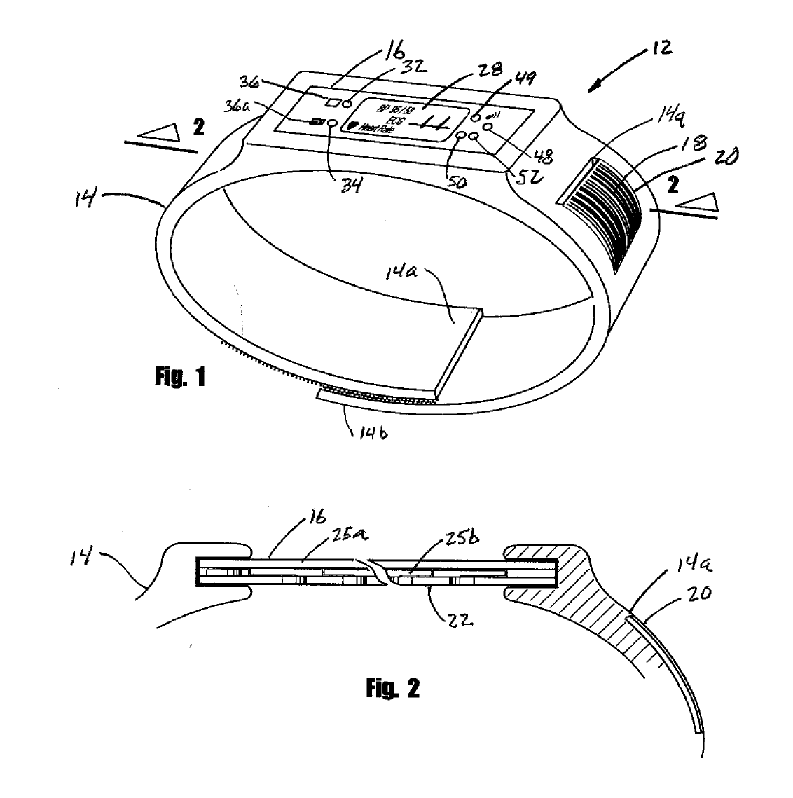

Referring to the drawings and particularly to FIGS. 1 through 5B, one form of the wireless physiological signal monitoring device of the invention for measuring physiological signals is there shown and generally identified by the numeral 12. Monitoring device 12 here comprises an adjustable, waterproof connector band 14 for interconnection with an appendage of the patient, such as the wrist, or ankle, and a vital sign monitor assembly 16 that is connected to the connector band in the manner shown in FIG. 1.

As previously mentioned, the monitoring device of the invention provides an identification system that is both digital and physical. Accordingly, the connector band 14 includes a data receiving slot 14a for removably receiving a printout of patient identification data. For example, in a hospital setting a printout 18 having a barcode, an identification number, a list of patient allergies and similar data can be inserted into slot 14a and can be viewed through a window 20.

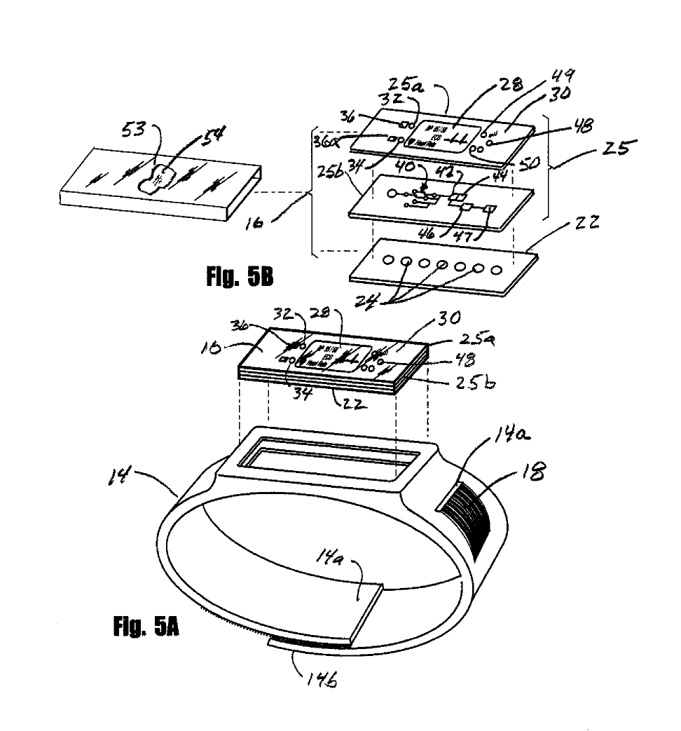

Vital sign monitor assembly 16, which is operably associated with a wireless network (see FIG. 8), includes a base portion 22 for interfacing with the patient. As best seen in FIGS. 4 and 5B of the drawings, base portion 22 is provided with a plurality of conventional sensors 24 for measuring vital signals from the patient, such as the patient's body temperature, blood pressure, electrocardiogram (ECG), heart rate, sleeping patterns, dehydration, blood oxygen levels, respiration and the like.

Operably associated with base portion 22 is an upper portion that is generally designated in FIG. 5B by the numeral 25. Upper portion 25 here comprises two levels 25a and 25b. Level 25a here includes a conventional digital touchscreen interface 28 and a component support surface 30. Carried by support surface 30 of level 25a are continuous colored light sources 32 and 34 that are programmed to illuminate the digital touchscreen interface 28 with a selected color that identifies a particular patient characteristic. For example, light sources 32, which can take the form of a conventional light emitting diode (LED), can be programmed to display pink or blue to represent the patient's gender, while light source 34, which can also take the form of a conventional light emitting diode (LED), can be programmed to indicate, with a specially selected color, a particular patient affliction, such as infant jaundice, patient allergies and like afflictions.

Touch screen sensors, such as digital touchscreen interface 28, function to detect the location of an object (e.g. a finger or a stylus) applied to the surface of a touch screen display or the location of an object positioned near the surface of a touch screen display. Examples of touch screen sensors that can be used in the device of the present invention include capacitive sensors, resistive sensors, and projected capacitive sensors. Also carried by level 25a is a conventional battery 36 that is operably associated with the colored light sources and with the digital touchscreen interface. Operably associated with battery 36 is a battery level indicator 36a for indicating the degree of charge of the battery.

Carried by level 25b is a suitably designed printed circuit 40 and a controller 42 that includes a conventional microprocessor 44 (FIG. 5B). Controller 42, which is operably associated with the printed circuit 40 and with the sensors 24, functions to receive and process the vital signals from the patient as measured by the sensors. In a conventional manner well understood by those skilled in the art, microprocessor 44 uses algorithms to translate these signals into viewable data. In a manner to be described in greater detail hereinafter and as illustrated in FIG. 5B of the drawings, a first wireless component, here shown as a conventional transducer 46, which is also operably associated with the controller 42 transmits the vital signals over a wireless network to a secure cloud server and system (see FIG. 8). A second wireless component, shown here as a conventional antenna 47, is operably associated with transducer 46 and cooperates therewith to transmit the vital signals over the wireless network.

As illustrated in FIGS. 1 and 5A of the drawings, also carried by level 25a of upper portion 25, is a conventional environmental sensor 48 for measuring external factors that exist proximate the device. Such external factors can include room temperature, humidity and the existence of airborne contaminants. Additionally, a conventional microphone 49 and a conventional speaker 50 are carried by level 25a (FIG. 1). Microphone 49, in cooperation with speaker 50, functions to broadcast to the caregiver selected vital signals of the patient as measured by the sensors. Further, speaker 50 functions to alert the caregiver to sounds emanating from a location proximate the patient, such as a baby crying, articles falling and other unusual, or alarming extraneous noises. Additionally, when desired, speaker 50 has the capability of playing sounds, such as lullabies, that are uploaded through the cloud system.

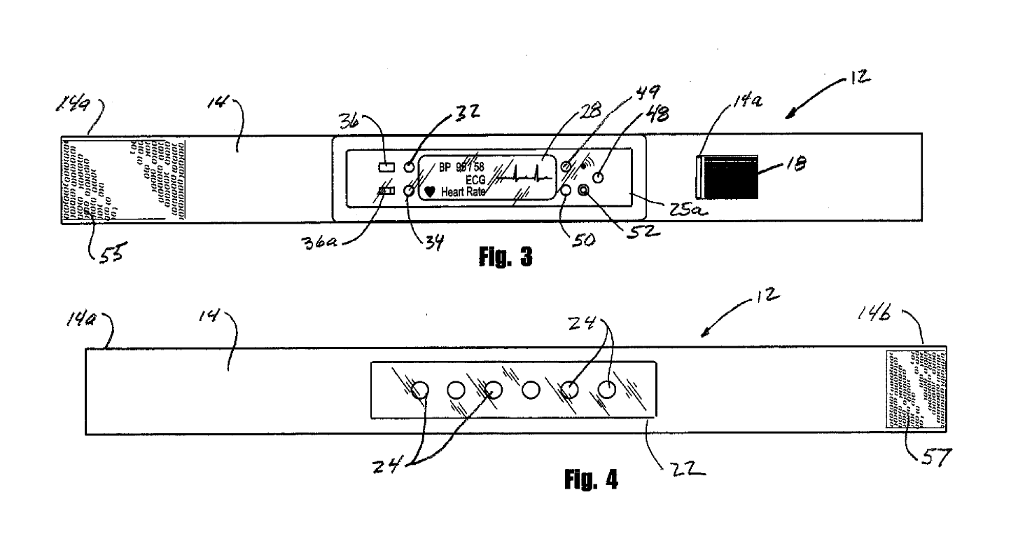

As illustrated in FIGS. 1 and 3 of the drawings, level 25a also carries a colored light source shown here as a warning LED 52. Warning LED 52, which is operably associated with the controller 42, is programmed to provide a continuous light display of either red or green depending on whether the patient's vitals are normal, or are a cause for concern.

In order to completely and securely waterproof the vital sign monitor assembly 16, a scratch resistant, waterproofing covering 53 that is adapted to sealably encapsulate the assembly is provided (see FIG. 5B). With the waterproofing covering in place about assembly 16 and, in light of the fact that the connector band 14 is waterproof, the entire device is rendered substantially waterproof, ensuring that patient vital signals can continue to be read even during water activities, such as bathing. Additionally, this important waterproofing feature of the invention permits the device to be easily sanitized through avenues such as soaking in a sanitizing solution without adversely affecting any component of the device.

Another important feature of the apparatus of the invention resides in the provision of a combination ultraviolet (UV), infrared (IV) protective film 54 (see FIG. 5) that comprises a part of the upper portion 25 of the vital sign monitor assembly 16. This protective film, which is carried by waterproof covering 53, functions to protect the sensors 24 that measure the patient's vital signs from harmful radiation so that they will effectively operate in virtually any environment. In this regard, since preemies are very often placed within incubators having relatively high temperatures, it is important that the sensors, and particularly the temperature-reading sensors, are suitably protected from damage so that they are able to properly function even in incubators exhibiting relatively high temperatures. With the protective film 54 in position in the manner shown in FIG. 5B of the drawings, the temperature sensors can reliably function to accurately determine the patient's temperature.

As previously mentioned, waterproof connector band 14 is readily adjustable. More particularly, in the present form of the invention, the connector band is provided with a first end 14a having a multiplicity of small loops 55 and with a second end 14b having a multiplicity of small hooks 57 that can be releasably interconnected with the loops 55. With this construction, the connector band can be easily adjusted to accommodate the size of the patient appendage to which the device is to be connected.

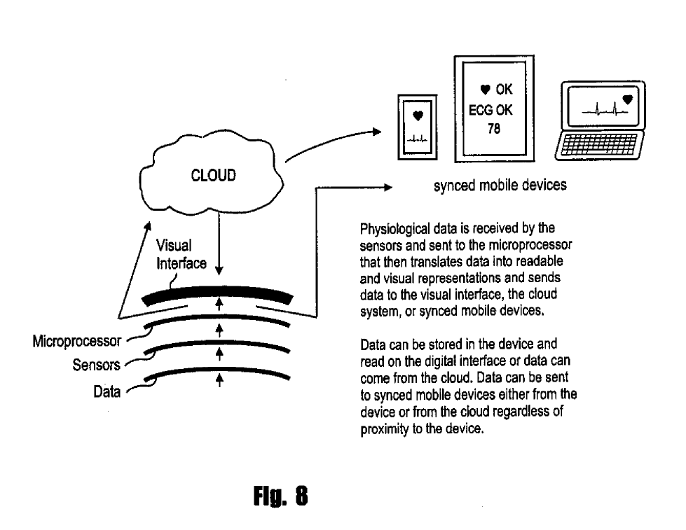

Turning now to FIGS. 7A through 7D, FIG. 7A shows in schematic form the vital sign monitor assembly 16 of the embodiment of the invention illustrated in FIGS. 1 and 2 of the drawings wherein each of the base portion 22 and levels 25a and 25b of the upper portion 25 are of a substantially rigid construction. FIG. 7B shows in schematic form an alternate embodiment of the monitor assembly wherein the base portion 62 as well as each of the levels 65a and 65b of the upper portion are of a pliable, yieldably deformable construction. FIG. 7C shows in schematic form yet another embodiment of the monitor assembly wherein the base portion 66 is of a pliable, yieldably deformable construction and levels 67a and 67b of the upper portion are of a substantially rigid construction. FIG. 7D shows in schematic form still another embodiment of the monitor assembly wherein the base portion 68 and the intermediate level 69b of the upper portion of the device is of a substantially rigid construction, while the upper level 69a of the upper portion is of a pliable, yieldably deformable construction. One advantage of the embodiment shown in FIGS. 7B and 7C is that the yieldably deformable base portions, which are in engagement with an appendage of the patient, will readily conform to the appendage and provide a comfortable interface therewith. One advantage of the embodiments shown in FIGS. 7B and 7D is that the uppermost level of the upper portion, which in these embodiments is yieldably deformable, will absorb accidental impact without damaging the monitor assembly.

As previously mentioned, the primary purpose of the device of the invention is the wireless monitoring of physiological signals from infants and small children. However, it is to be appreciated that the device can also be used for the wireless monitoring of physiological signals from adults and other animals. In use, the device can be worn at any location along the patient's arm or leg, with the wrist or ankle being the preferable location. Since the device is readily adjustable (see FIG. 1) it can readily accommodate different arm and leg circumferences.

Data that is collected via the sensors 24 is accessible both on the device's visual interface and, as illustrated in FIG. 8 of the drawings, can be automatically uploaded to a secure cloud server. Similarly, when desired, the device can be programmed to send the data to specified devices without use of the cloud server, thereby permitting the viewing of data both historically and in real time.

As indicated in FIG. 8, the physiological data is uploaded in a manner well understood by those skilled in the art via wireless channels such as Wi-Fi and radio signals and preferably is programmed to use two or more different channels in order to ensure consistent data feed. More particularly, in operation, the physiological data, which is received by the sensors 24, is sent to the microprocessor 44, which translates the data into a readable form. The data is then sent to the visual interface 25a and then on to the cloud system or to the synced mobile devices. If for some reason wireless transmission of data is not possible, the device can be connected directly to other mobile devices or to traditional healthcare machines, such as ECG monitors, and can then be read through such monitors.

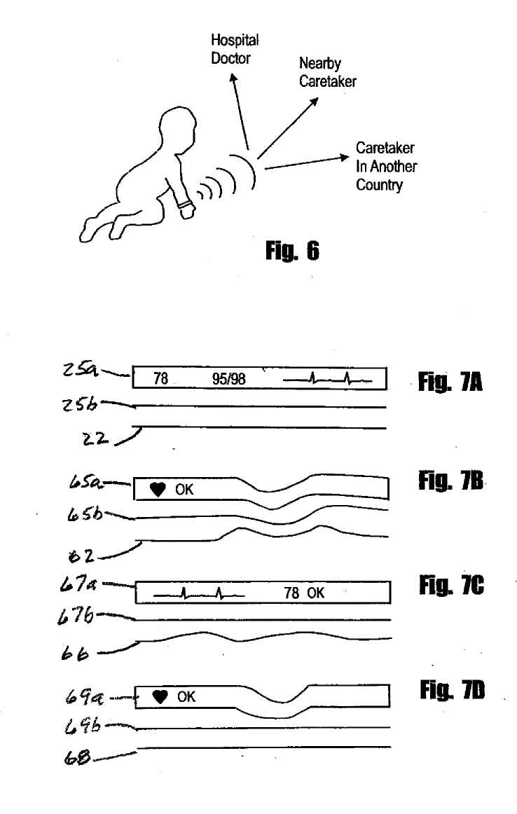

Data is saved indefinitely on the secure cloud server and within the device itself for a predetermined length of time, (e.g. hours, days, months, etc.) in order to ensure that the data is safely maintained in the event of an outage in the wireless transmission. As indicated by FIG. 8 of the drawings, the data is continuously reviewed by both the device and the cloud server, and both systems alert the caregiver if certain thresholds are met. This will allow caretakers, both foreign and domestic (see FIG. 6), to glean and transmit important information from historical data and patterns. It will also permit the hospital and the patient's doctor to continuously monitor at-risk infants and to conduct research on various afflictions such as sudden infant death syndrome. As indicated in FIG. 6 of the drawings, the patient data can be forwarded to the hospital doctor, to a nearby caretaker, or even to a caretaker in another country. Also, the microprocessor 44 can be imbued with intelligence software that allows the system to adjust the patient baselines (i.e., it may be observed that patient consistently has a higher blood pressure than normal and adjusts to account for such condition).

Advantageously, the data retrieved by the device is accessible regardless of physical proximity to the monitoring device and both real-time and historical data is available. The constant monitoring of the infant, rather than monitoring a specific signal as a result of an issue, allows pediatric health care to become much more preemptive and efficient. In practice, use of the device of the invention will improve quality and efficiency of pediatric care in both home and clinical settings. Use of the device will also lower incidents of incorrect paper records or mistakes, such as not recognizing medication allergies.

As previously discussed, the digital interface 28 of the device can be customized to show different real-time data, such as current heart rate or blood pressure. The digital interface 28 can also be programmed to, for example, display pink or blue to represent the infant's gender. Another green or red light provided on the digital interface 28 continuously signals to the caregiver whether the patient's vital signs are normal, or are a cause for concern. The digital interface 28 also displays current battery charge levels, and alerts the caregiver when the device requires a battery charge or replacement.

As indicated in FIG. 8 of the drawings, in operation, physiological data received by the sensors 24 and sent to the microprocessor 44, which translates the data into a readable form in visual representations, is sent to the visual interface and to either the cloud, or to a selected one of several synced mobile devices. The physiological data can be stored in the device and read on the digital interface 28, or the data can be transmitted from the cloud. Similarly, data can be sent to the synced mobile devices either from the device, or from the cloud without regard to proximity to the device.

Having now described the invention in detail in accordance with the requirements of the patent statutes, those skilled in this art will have no difficulty in making changes and modifications in the individual parts or their relative assembly in order to meet specific requirements or conditions.

Such changes and modifications may be made without departing from the scope and spirit of the invention, as set forth in the following claims.

I claim:

1. A wireless physiological signal monitoring device for monitoring the vital signs of a patient, comprising:
(a) a connector band for interconnection with the patient;
(b) a vital sign monitor assembly connected to said connector band, said vital sign monitor assembly being operably associated with a wireless network and including a base portion for interfacing with the patient, said base portion having at least one sensor for measuring vital signals from the patient and an upper portion operably associated with said base portion, said upper portion including:
(i) digital touchscreen interface;
(ii) a continuous colored light source programmed to illuminate said digital touchscreen interface with a selected color that identifies a particular patient characteristic;
(iii) a controller including a microprocessor operably associated with said at least one sensor of said base portion for receiving and processing the vital signals from the patient as measured by said at least one sensor;
(iv) a speaker operably associated with said controller for broadcasting selected vital signals of the patient as measured by said at least one sensor;
(v) a battery operably associated with said colored light source, with said speaker and with said digital touchscreen interface; and
(vi) a first wireless component operably associated with said controller for transmitting said vital signals over said wireless network; and
(c) a waterproof covering encapsulating said vital sign monitor assembly for waterproofing said vital sign monitor assembly.

2. The wireless physiological signal monitoring device as defined in claim 1, in which said connector band includes an identification area in which a printout of patient physical identification data is affixed.

3. The wireless physiological signal monitoring device as defined in claim 1, in which said upper portion of said vital sign monitor assembly further includes a microphone for detecting sounds emanating from a location proximate the patient, said microphone being operably associated with said speaker.

4. The wireless physiological signal monitoring device as defined in claim 1, in which said vital sign monitor assembly further includes an environmental sensor for measuring external factors existing proximate said device.

5. The wireless physiological signal monitoring device as defined in claim 1, in which said vital sign monitor assembly further includes a second wireless component operably associated with said controller for transmitting said vital signals over said wireless network.

6. The wireless physiological signal monitoring device as defined in claim 1, in which said base portion of said vital sign monitor assembly is yieldably deformable.

7. The wireless physiological signal monitoring device as defined in claim 1, in which said upper portion of said vital sign monitor assembly further includes an infrared, ultra violet protective film.

8. The wireless physiological signal monitoring device as defined in claim 1, in which said digital touchscreen interface of said upper portion of said vital sign monitor assembly is yieldably deformable.

9. A wireless physiological signal monitoring device for monitoring the vital signs of a patient, comprising:
(a) a connector band for interconnection with an appendage of the patient, said connector band having a data receiving slot for removably receiving selected patient identification data; and
(b) a vital sign monitor assembly connected to said connector band, said vital sign monitor assembly being operably associated with a wireless network and including a base portion for interfacing with the patient, said base portion having at least one sensor for measuring vital signals from the patient; and an upper portion operably associated with said base portion, said upper portion including:
(i) a digital touchscreen interface;
(ii) a continuous colored light source programmed to illuminate said digital touchscreen interface with a selected color that identifies a particular patient characteristic;
(iii) a microphone for detecting sounds emanating from a location proximate the patient;
(iv) a speaker operably associated with said microphone for broadcasting sounds detected by said microphone;
(v) a battery operably associated with said colored light source, with said microphone, with said speaker and with said digital touchscreen interface;
(vi) a controller including a microprocessor operably associated with said at least one sensor of said base portion for receiving and processing the vital signals from the patient as measured by said at least one sensor;
(vii) a first wireless component operably associated with said controller for automatically transmitting said vital signals over said wireless network; and
(viii) an environmental sensor for measuring external factors existing proximate said device.

10. The wireless physiological signal monitoring device as defined in claim 9, further comprising a waterproof covering encapsulating said vital sign monitor assembly for waterproofing said assembly.

11. The wireless physiological signal monitoring device as defined in claim 9, in which said upper portion of said vital sign monitor assembly is yieldably deformable.

12. The wireless physiological signal monitoring device as defined in claim 9, in which said upper portion further includes a battery level indicator operably associated with said battery.

13. The wireless physiological signal monitoring device as defined in claim 9, in which said vital sign monitor assembly further includes a second wireless component operably associated with said controller for automatically transmitting said vital signals over said wireless network.

14. A wireless physiological signal monitoring device for monitoring the vital signs of a patient, comprising:
(a) a connector band for interconnection with the wrist of the patient, said connector band having a data receiving slot for removably receiving a printout of patient identification data;
(b) a vital sign monitor assembly connected to said connector band, said vital sign monitor assembly being operably associated with a wireless network and including a base portion for interfacing with the patient, said base portion having a plurality of sensors for measuring vital signals from the patient; and an upper portion operably associated with said base portion, said upper portion including:
(i) a pliable digital touchscreen interface;
(ii) a continuous colored light source programmed to illuminate said digital touchscreen interface with a selected color that identifies a particular patient characteristic;
(iii) a microphone for detecting sounds emanating from a location proximate the patient;
(iv) a speaker operably associated with said microphone for broadcasting sounds detected by said microphone;
(v) a battery operably associated with said colored light source, with said microphone, with said speaker and with said digital touchscreen interface;
(vi) an environmental sensor for measuring external factors existing proximate said device;
(vii) a controller including a microprocessor operably associated with said at least one sensor of said base portion for receiving and processing the vital signals from the patient as measured by said at least one sensor;
(viii) a first wireless component operably associated with said controller for transmitting said vital signals over said wireless network; and
(ix) an infrared, ultra violet protective film; and
(c) a scratch resistant, waterproofing covering encapsulating said vital sign monitor assembly for waterproofing said vital sign monitor assembly.

15. The wireless physiological signal monitoring device as defined in claim 14, in which said upper portion further includes a battery level indicator operably associated with said battery.

16. The wireless physiological signal monitoring device as defined in claim 14, in which said base portion of said vital sign monitor assembly is yieldably deformable.

17. The wireless physiological signal monitoring device as defined in claim 14, in which said vital sign monitor assembly further includes a warning light emitting diode that is operably associated with said controller for providing a continuous light display of either red or green depending on whether the patient's vitals are normal, or are a cause for concern.
